(19) United States
(12) Patent Application Publication
(10) Pub. No.: US 2005/0197561 A1
(43) Pub. Date: Sep. 8, 2005

(54) System for detecting symptoms, determining staging and gauging drug efficacy in cases of Parkinson's disease

(76) Inventors: Catherine L. Elsinger, Wauwatosa, WI (US); Stephen M. Rao, Shorewood, WI (US)

Correspondence Address: John Horn;Neurognostics c/o Techstar, Suite 250, 756 N. Milwaukee St., Milwaukee, WI, 53202, US

(21) Appl. No.: 10/793,995

(22) Filed: Mar. 5, 2004

(51) Int. Cl. A61B 5/05

(52) U.S. Cl. 600/410

(57) ABSTRACT

A system for using functional magnetic resonance imaging (fMRI) for detecting symptoms indicative of Parkinson's disease, diagnosing Parkinson's disease and gauging the efficacy of medications used in treating Parkinson's disease. The system includes process steps involving activating a selected region of the brain which may be affected by Parkinson's disease using a delayed response motor sequence type task, concurrently acquiring task-active MRI data responsive to the task, comparing the patient's task-active MRI data to reference data derived from a database of task-active data from healthy individuals and detecting whether the patient has symptoms related to Parkinson's disease. The severity of the patient's symptoms and the staging of the disease may also be determined. Also, a medication may be administered to the patient and the efficacy of the medication may gauged based on the severity of the patient's symptoms on and off of medication.

### FIELD OF THE INVENTION

The present invention relates to systems for use in detecting symptoms of neurodegenerative disorders and more specifically to using functional magnetic resonance imaging (fMRI) for detecting symptoms, staging and gauging drug efficacy in cases of Parkinson's disease.

### BACKGROUND OF THE INVENTION

Parkinson's Disease (PD) is a progressive and incurable neurological disease most often beginning in the sixth decade of life. PD afflicts an estimated 4 million people worldwide is the most common neurodegenerative movement disorder affecting more than 0.1% of the population over 40 years of age. Annual health care costs in the United States associated with PD have been estimated to be in excess of $6B. The core motor features of PD include bradykinesia (slowness of movement), akinesia (difficulty initiating movement), rigidity, tremor, and loss of postural reflexes. The progressive neurodegeneration is the result of a steep decline in the number of neurons in the substantia nigra pars compacta (SNpc); this brain structure is responsible for generating dopamine (DA). When the amount of DA produced falls below 80% of normal, disruption occurs in various DA mediated brain circuits that involve the basal ganglia and medial premotor cortex. These brain regions are critically involved in higher order aspects of movement control, cognitive functioning (e.g., memory, attention), and emotions. PD patients indicate a deficit in generating complex sequences of movements in the absence of an environmental cue. This deficit is present at the level of organizing sequential finger movements of the same effector and at the level of coordinating multiple effectors or body segments. Patients show particular deficits in performing sequential and simultaneous movements that require added planning, execution time or timing processes.

At the present time there are no treatments that have been shown to slow the progression of this debilitating disease. However, a number of FDA-approved therapeutic interventions (pharmaceutical, surgical and physiological) have become available for the management of motor and cognitive complications associated with PD. The most common and effective approach for treating PD symptoms involves administration of medications (Levodopa, or L-dopa) that replace deficient dopamine (DA) and/or act as DA receptor agonists. While these treatments substantially benefit PD patients, prolonged use of dopamine replacement therapy has been associated with the development of adverse effects, such as painful dyskinesias, fluctuations in motor symptoms, and hallucinations. Furthermore, the advanced stages of PD do not respond to dopamine replacement therapy. Thus, after 5 to 10 years of treatment, most PD patients develop disabilities that cannot adequately be controlled with available medical treatment. More effective and longer lasting treatments need to be developed that alter the disease course in addition to managing symptoms. However, existing standard clinical outcome measures essential for evaluating PD treatments such as the Unified Parkinson's Disease Rating Scale (UPDRS) and the Core Assessment Program for Surgical Intervention Therapies in Parkinson's Disease (CAPSIT-PD) suffer from relatively low reliability and sensitivity.

Positron emission tomography (PET) and single photon emission computed tomography (SPECT) represent two in vivo neuroimaging techniques for assessing presynaptic dopamine transporter (DAT) distribution in humans. PET and SPECT techniques both involve the use of radiolabelled molecules to image nigrostriatal neuronal loss in PD. Unfortunately, the results of many PET and SPECT studies have indicated inconsistencies in the correlations between DAT ligand binding and PD motor dysfunction. These inconsistencies have been variously attributed to vulnerability of PET and SPECT to confounding effects of age, tobacco usage, and use of antiparkinsonian medications. However, the most important limitation of these imaging measures is that they record resting brain activity and they do not measure the brain's response to cognitive motor behaviors that stress neural systems directly affected by this disease. Further, PET and SPECT require the injection of a radioisotope and present safety limitations in terms of the number of studies that can be administered to a given patient over a short period of a time, thereby limiting ability to monitor drug efficacy. Further, PET and SPECT also require the on-site or nearby installation and maintenance of a cyclotron (due to the short half life of radioisotopes used to measure cerebral blood flow), thus generally limiting the installed base of available machines.

In U.S. Pat. No. 4,931,270 to Horn et al. a method is disclosed for detecting dopaminergic diseases using radiolabelled dopamine receptor ligands. Abnormalitites in the distribution of a dopamine D.sub.2 receptors are detected by administering an amount of a .sup.18 F-radiolabelled compound to a patient sufficient for PET imaging purposes, using PET to form at least one image showing the distribution of the radiolabelled compound within the patient and determining the normality of the concentrations or distribution by comparing the image of the patient with an image showing the normal concentrations and distributions of the receptors in other patients. The abnormalities detected are taken as an indication of the presence of disease.

fMRI is a neuroimaging technology which has been used in researching functional aspects of central nervous system disorders. fMRI is an application of conventional nuclear or MRI technology in which functional brain activity is detected usually in response to an activation task being performed by a patient. fMRI is capable of detecting localized event-related brain activity and changes in this activity over time. Its principal advantages are its strong spatial and temporal resolution and, as no isotopes are used, a virtually unlimited number of scanning sessions that can be performed on a given subject, making within subject designs feasible. fMRI operates by detecting increases in cerebral blood volume that occur locally in association with increased neuronal activity. A widely used fMRI method for detecting brain activity is based upon the blood oxygenation level dependent (BOLD) response. The BOLD signal arises as a consequence of a ‘paradoxical’ increase in blood oxygenation, presumably due to increased local blood flow in excess of local metabolic demand and oxygen consumption following neuronal activity. An increase in blood oxygenation results in increased field homogeneity (increase in T2 and T2*), less dephasing of spins, and increased MR signal on susceptibility-weighted MRI images.

### SUMMARY OF THE INVENTION

The present invention comprises a system for detecting symptoms related to Parkinson's disease, diagnosing and monitoring the progression of the disease and assessing the efficacy of medications in treating the disease. The system uses an MRI scanner to implement a functional magnetic resonance imaging (fMRI) scanning process in which a delayed response motor sequencing task is performed by a patient during an MRI scan. The MRI scanner generates a time image series of MRI scan data showing functional activity in the brain generated by the delayed response motor sequencing task.

The delayed response motor sequencing task is employed in order to engage processes related to movement planning and stimulate activity in regions of the brain such as the basal ganglia and frontal cortex including medial premotor area (SMA) regions directly affected by Parkinson's disease. In the preferred embodiment the delayed response motor sequencing task involves visually presenting a numerical sequence to a patient, allowing for a delay or planning period and then having the patient tap out this sequence on a keyboard. The complexity of the sequence and the length of the delay period are preferably varied. Delayed response motor sequencing related MRI data indicative of the functional MRI brain activity of the patient responsive to the task is acquired and recorded. The task-active MRI data is analyzed by making comparisons between this data for the individual patient and standards for functional brain activity responsive to delayed response motor sequencing tasks derived from reference data from healthy patients. On the basis of these comparisons symptoms related to Parkinson's disease may be detected and the presence and progress of Parkinson's disease in the patient may be diagnosed.

In a further embodiment a medication intended to address symptoms related to Parkinson's disease is administered to the patient. The resulting task-active MRI data from the patient is analyzed and compared with delayed response motor sequencing task-activated data elicited from the patient when not on medication. The patient's data may also be compared with reference data derived from a reference database including delayed response motor sequencing activity from healthy subjects and subjects known to be afflicted with Parkinson's disease. The effectiveness of the medication can then be evaluated based on the relative severity of the symptoms detected in said patient.

It is an object of the present invention to provide a system for detecting the symptoms of Parkinson's disease during the development of the disorder using fMRI technology.

It is a further object of the present invention to provide a system for accurately assessing the severity of the symptoms of Parkinson's disease and assessing the staging of the disease using fMRI technology.

It is another object of the present invention to provide a system for gauging the efficacy of drugs in treating Parkinson's disease using fMRI technology.

It is a yet further object of the present invention to provide a system for detecting the symptoms of Parkinson's disease and their severity in an efficient, consistent and reliable manner.

It is yet another object of the present invention to provide an activation task for use in fMRI studies for stimulating brain activity in regions of the brain known to be affected by Parkinson's disease.

### BRIEF DESCRIPTION OF THE DRAWINGS

FIG. 1 provides a diagrammatic illustration of a magnetic resonance imaging machine and its major components as adapted for performing functional magnetic resonance imaging studies.

FIG. 2 provides a flowchart illustrating the operative process for detecting the symptoms, diagnosing and determining the staging of Parkinson's disease in accordance with the present invention.

FIG. 3 provides a flowchart illustrating the operative process for detecting the symptoms and gauging the efficacy of medications intended to treat Parkinson's disease in accordance with the present invention.

FIG. 4 provides a flowchart illustrating the sub-steps of the delayed response motor sequencing task in accordance with the present invention.

### DETAILED DESCRIPTION OF THE INVENTION

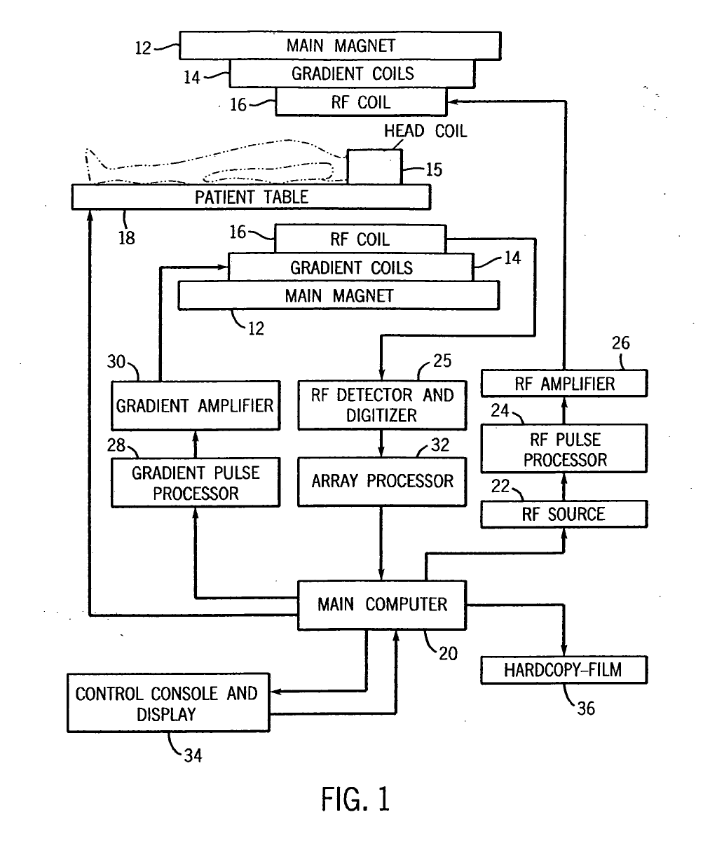

Referring now to FIG. 1, the basic components of a magnetic resonance imaging (MRI) machine 10 are shown. The main magnet 12 produces a strong Bₒ field for the imaging procedure. Within the magnet 12 are the gradient coils 14 for producing a gradient in the Bₒ field in the X, Y, and Z directions as necessary to provide frequency discrimination. A head coil 15 is also used to improve accuracy and resolution for studies involving the brain. Within the gradient coils 14 is a radio frequency (RF) coil 16 for producing RF pulses and the B₁ transverse magnetic field necessary to rotate magnetic spins by 90° or 180°. The RF coil 16 also-detects the return signal from the spins within the body and supplies these signals to the RF detector and digitizer 25. The patient is positioned within the main magnet by a computer controlled patient table 18. The scan room is surrounded by an RF shield, which prevents the high power RF pulses from radiating out through the hospital and prevents the various RF signals from television and radio stations from being detected by the imager. The heart of the imager is the computer 20 that controls the components of the imaging system. The RF components under control of the computer include the radio frequency source 22 and pulse programmer 24. The source 22 produces a sine wave of the desired frequency. The pulse programmer 24 shapes the RF pulses into apodized sinc pulses. The RF amplifier 26 greatly increases the power of the RF pulses. The computer 20 also controls the gradient pulse programmer 28 which sets the shape and amplitude of each of the three gradient fields. The gradient amplifier 30 increases the power of the gradient pulses to a level sufficient to drive the gradient coils 14. In most systems an array processor 32 is also provided for rapidly performing two-dimensional Fourier transforms. The computer 20 off-loads Fourier transform tasks to this faster processing device. The operator of the imaging machine 10 provides input to the computer 20 through a control console 34. An imaging sequence is selected and customized by the operator from the console 34. The operator can see the MRI images on a video display located on the console 34 or can make hard copies of the images on a film printer 36.

A General Electric Signa EXCITE 3.0 Tesla MRI scanner is preferably used for performing whole-brain imaging and implementing the present invention although any of a number of commercial MRI scanners having 3.0 or 1.5 (or less) Tesla fields could be used. Echo-planar (EP) images are collected using a single-shot, blipped, gradient echo EP pulse sequence; echo time (TE)=40 ms, with 40 ms of image acquisition time. The interscan period (TR) is 2 seconds. Typical image resolution will be 64×64 voxels with a 24 cm field of view (FOV). Twenty-two contiguous sagittal 6 mm thick slices are selected in order to provide coverage of the entire brain (3.75×3.75×6 mm typical voxel size). An additional 6 images are added to the beginning and end of the run to accommodate the delayed rise of the hemodynamic response. Prior to functional imaging, 124 high-resolution spoiled GRASS (gradient-recalled at steady-state) sagittal anatomic images [TE=5 ms; TR (repetition time)=24 ms, 40° flip angle, NEX (number of excitations)=1, slice thickness=1.2, FOV=24 cm, matrix size=256×128] are acquired on each subject. These images serve as the high-resolution anatomic images that allow precise localization of functional activity and co-registration. Visual stimuli are computer-generated and rear-projected (video projector) on an opaque screen located at the subject's feet. Subjects view the screen through prism glasses attached to the head coil. Corrective lenses can be provided if necessary. The viewing distance is about 220 cm. A non-ferrous three-button key-press (keyboard) device made from force-sensing resistors is used to record responses, accuracy and reaction time. To provide precise time synchronization between the presentation of visual stimuli and the scan sequence, a trigger signal coincident with the acquisition of each MR image is fed into the computer controlled video display.

Foam padding is preferably used to limit head motion within the head coil. Head movement, typically subvoxel (<2 mm), is viewed in cine format. The image time series is spatially registered to minimize the effects of head motion and a 3D volume registration algorithm is used align each volume in each time series to a fiducial volume through a gradient descent in a nonlinear least squares estimation of six movement parameters (3 shifts, 3 angles).

The delayed response motor sequencing activation task combines simple and complex motor sequences presented in random order. Briefly, each trial begins with the presentation of a preparatory cue displaying a numerical sequence. The cue is followed by a delay period that may be several seconds in length characterized by movement planning on the part of the patient. A “go” cue is then presented signaling the patient to perform a motor sequence corresponding to the preparatory cue. Motor sequence tasks may be simple or complex. The preparatory cue serves as a prompt and contains full information about the upcoming sequence. Participants are required to hold this sequence in working memory throughout the delay period, plan the appropriate response during this period and execute the sequence when the (non-informative) go signal appears. The simple sequence task consisted of five repetitive movements involving a single digit. Complex movement sequences may involve heterogeneous sequences which may use multiple digits and multiple transitions. The imaging analyses comprise comparisons of the intensity and extent of regional cerebral activity arising with respect to the activation task. The fMRI data analyses generate impulse response functions (IRFs) based on correct trials for the simple and complex sequences within and across groups on a voxelwise basis. These are also summarized by an averaged IRF response for the selected regions of interest (ROIs) affected by Parkinson's disease [e.g., subregions of the basal ganglia (Globus Pallidus internal segment (GPi), Globus Pallidus external segment (GPe), caudate, putamen) and thalamus, as well as subregions of the frontal cortex (primary motor cortex, lateral premotor, pre-SMA (Supplementary Motor Area), SMA, anterior cingulate, dorsolateral cortex)].

The exact activation task involves the subject performing finger key presses in response to numerical sequences that are presented visually on an image (computer) screen. The index, middle, and ring fingers of the patient's right hand will ordinarily be placed, respectively, over the left (“1”), middle (“2”), and right (“3”) keys of a small keyboard. Prior to trial onset, subjects view a black crosshair located in the center of a blank white screen. Trials are presented in a randomized fashion both within and across imaging runs. A trial begins with the presentation of a 5-digit preparatory cue (printed in black) that appears horizontally in the middle of the image screen for 2 seconds. This cue provides the subject with full information about the motor sequence to be performed in the future. During the delay period the preparatory cue is replaced with a row of x's, which act as visual placeholders. After a variable delay (4 or 6 seconds) designed to minimize anticipatory effects, a “go” cue (printed in green), is presented for 4 seconds, signaling the subject to perform the motor sequence as quickly and accurately as possible. This is followed by a variable inter-trial interval (2 or 4 sec) prior to the next preparatory cue.

The complexity of the motor sequence to be performed and the delay between preparatory cue and go signal are varied. The dependent measures collected for each trial include reaction time (RT), accuracy, and movement time (MT). Half of the trials are Complex (C) and half of the trials are Simple (S). Subjects perform one of 6 possible heterogeneous sequences (12131, 23231, 32321, 13121, 21313, 31212) or one of 3 different homogeneous sequences (11111, 22222, 33333) at the “Go” cue. These sequences are chosen because they tend to maximize differences in both surface structure and number of finger transitions. In all cases, a delay period of 4 or 6 seconds separates the cue and the go signal. During this period the cue sequence must be maintained in memory. The variable delay between the preparatory cue and the go signal, and the variable inter-trial interval delay accomplish two goals. The introduction of these delay periods minimizes subject expectation by reducing temporal regularity in the task and also introduces a random interstimulus interval (ISI) that aids in the deconvolution analysis. A typical imaging run consists of 24 14-second trials, 12 12-second trials, 12 16-second trials, and a 12-second rest period at the beginning and end of each run. Each subject ordinarily undergoes four event-related functional imaging runs. Scanning is synchronized with the trial onset, with 6-8 images (12-16 seconds) acquired during each trial, for a total of 336 images per run (48 trials per run). Practice trials are administered outside the scanner and monitored for accuracy to ensure that the subject fully understands the task demands.

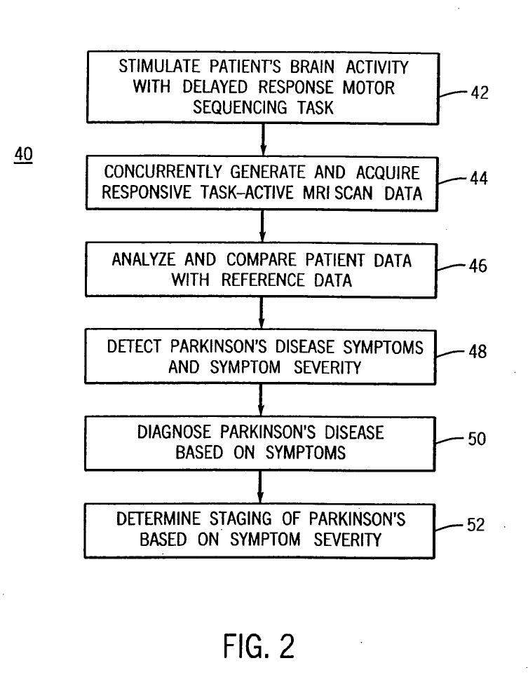

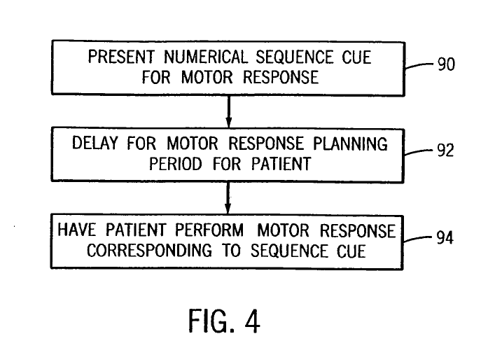

Referring now to FIGS. 2 and 4, the operative process 40 for detecting the symptoms, diagnosing and determining the staging of Parkinson's disease includes the steps 42, 44, 46, 48, 50 and 52. In step 42 the patient is stimulated using a delayed response motor sequencing task in order to generate activity in regions of the patient's brain such as the basal ganglia and frontal cortex and certain of their subregions that may be directly involved with Parkinson's disease. First, in step 90 a numerical sequence is visually presented to the patient on the basis of which the patient plans a motor response. A delay period is then provided in step 92 during which the subject plans a corresponding motor response. In step 94 a visual go signal is provided the patient thereafter taps out this response on a keyboard. Step 44 is performed concurrently with step 42 so that scanning and data acquisition takes place by the MRI machine as brain activity is stimulated in response to the delayed response motor sequencing task. In step 44 delayed response motor sequencing related MRI data is acquired and recorded by the MRI scanning system. The delayed response motor sequencing related MRI data is then analyzed in step 46 by making comparisons between the patient's delayed response motor sequencing related data, or indexes derived from this data, and reference data, indexes, or standards for functional brain activity responsive to delayed response motor sequencing tasks derived from MRI data collected from healthy patients and from patients known to be afflicted with Parkinson's disease. In step 48 the presence and associated severity (or absence) of one or more symptoms related to Parkinson's disease are detected based on these comparisons. Accordingly, in step 50 the patient is diagnosed as having or not having the disease in accordance with the symptoms detected and, if the patient is in fact diagnosed with the disease, in step 52 the staging (state of progression) of Parkinson's disease is determined based on the relative severity of said symptoms detected in the patient.

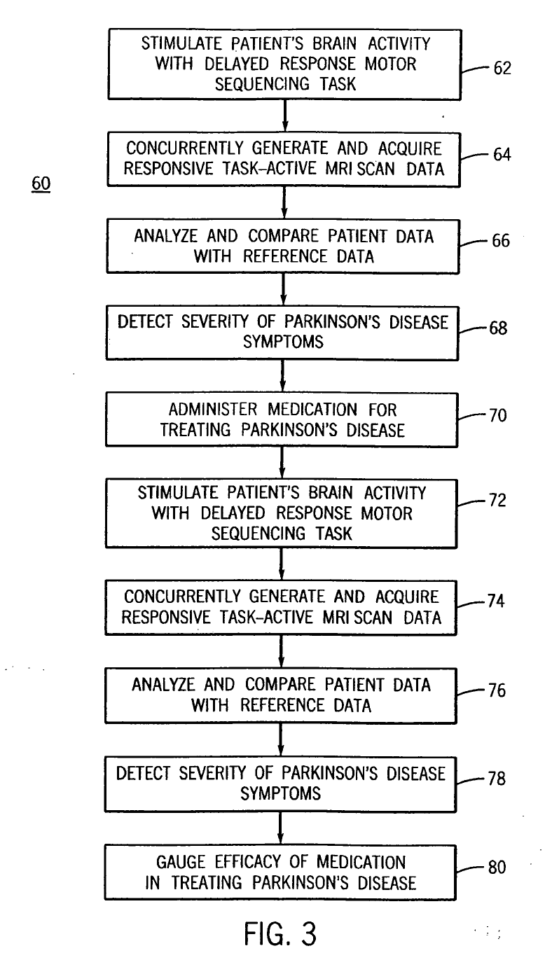

Referring now to FIG. 3, the operative process 60 for detecting the symptoms and gauging the efficacy of medications intended to treat Parkinson's includes the steps 62, 64, 66, 68, 70, 72, 74, 76, 78 and 80. Steps 62, 64, 66 and 68 are similar to steps 42, 44, 46 and 48 as described above and involve activating a selected region of the brain using an delayed response motor sequencing type task, concurrently acquiring task-active MRI data responsive to the delayed response motor sequencing task, comparing the patient's MRI data to reference data from healthy individuals and detecting the relative severity of the symptoms of Parkinson's disease in the patient. In step 70 a medication intended to treat Parkinson's disease is administered to the patient. Steps 72, 74, 76, and 78 are again similar to steps 42, 44, 46 and 48 as described above and involve activating a selected region of the brain using an delayed response motor sequencing type task, concurrently acquiring task-active MRI data responsive to the delayed response motor sequencing task, comparing the patient's MRI data to reference data from healthy individuals and detecting the relative severity of the symptoms of Parkinson's disease in the patient. However, in step 80 the effectiveness of the medication administered in step 70 is gauged based on the relative severity of the symptoms detected in the patient when under said medication and when not under said medication.

Several publicly available software programs such as AFNI (Medical College of Wisconsin in Milwaukee, Wis.) and BrainVoyager (Brain Innovation B.V. in Maastricht, Netherlands) have been developed that allow for whole-brain, 3D fMRI activation mapping and within- and between-subjects statistical comparisons and also include extensive statistical routines. Typically, all whole-brain fMRI data are converted to 4D data sets (time plus 3 spatial dimensions). Functional images are directly registered upon high resolution anatomical scans obtained in the same imaging session. Location and intensity of activation from individual or grouped data are translated into 3D proportionally measured, stereotaxic coordinates relative to the line between the anterior and posterior commissures.

Functional images are first time-locked to the events of interest (e.g., correct performance of a delayed response motor sequence). A software program such as 3dDeconvolve (AFNI) is used to estimate the system impulse response function (IRF). This program uses a sum of scaled and time-delayed versions of the stimulus time series, with the data itself determining (within limits) the functional form of the estimated response. The program yields the best linear least-squares fit for the following model parameters: constant baseline, linear trend in time series, and estimates the IRF for 7-9 images post-stimulus onset (14-18 sec.) for each condition relative to a baseline state. In a typical imaging run, approximately 33% of “trials” involve a baseline control condition to introduce “jitter” in the time series. Active trials are coded by condition and accuracy.

High resolution anatomical and functional images are linearly interpolated to volumes with 1 mm³ voxels, co-registered, and converted to stereotaxic coordinate space. Functional images are typically blurred using a 4 mm Gaussian full-width half-maximum (FWHM) filter to compensate for intersubject variability in anatomic and functional anatomy. Voxel-wise statistical analyses across fMRI 3-D data sets are achieved with 3dANOVA type models (applicable to both within- and between-subject designs). Instead of using the individual voxel probability threshold alone, probability thresholding is used in combination with minimum cluster size thresholding. The underlying principle is that true regions of activation will tend to occur over contiguous voxels, whereas noise has much less of a tendency to form clusters of activated voxels. By combining the two, the power of the statistical test is greatly enhanced. If desired the tradeoff between probability and cluster threshold can be adjusted to achieve the desired significance level. By iteration of the process of random image generation, Gaussian filtering (to simulate spatial correlation between voxels), thresholding, and tabulation of cluster size frequencies, a Monte Carlo simulation program such as AphaSim can be used to generate an estimate of the overall significance level achieved for various combinations of individual voxel probability threshold and cluster size threshold assuming spatially uncorrelated voxels.

While voxel-wise statistical analyses are easy to implement, they may distort information due to normal variations in cortical and subcortical topography. These differences become magnified when comparing brain activation patterns across groups of subjects (healthy vs. PD ON vs. PD OFF). In the preferred embodiment there are several regions and subregions of the brain that comprise specific regions of interest (ROIs) to be analyzed in detail including certain subregions of the basal ganglia (GPi, GPe, caudate, putamen) and thalamus, as well as subregions of the frontal cortex (primary motor cortex, lateral premotor, pre-SMA, SMA, anterior cingulate, dorsolateral cortex). As a part of the overall analyses three dependent values are calculated for each selected region of interest (ROI): (1) the number of activated voxels divided by the total number of voxels in the region, a measure of the spatial extent of the activated region, (2) the mean % area-under-the-curve (% AUC) of the activated voxels, a measure of the intensity of the activated region, and (3) a power function defined as the percent of activated voxels in an ROI multiplied by the mean % AUC, an index that combines spread and intensity information.

Although the invention has been described with reference to certain embodiments for which many implementation details have been described, it should be recognized that there are other embodiments within the spirit and scope of the claims and the invention is not intended to be limited by the details described with respect to the embodiments specifically disclosed.

1. In a functional MRI scanning process in which an activation task is performed during an MRI scan for the purpose of generating functional activity data, the process including the steps comprising:
a) stimulating a patient using an delayed response motor sequencing task in order to activate regions of the brain known to be affected by Parkinson's disease;
b) acquiring and recording a first set of delayed response motor sequencing related MRI data indicative of the functional MRI brain activity of the patient responsive to said delayed response motor sequencing task;
c) analyzing said delayed response motor sequencing related MRI data by making comparisons between said first delayed response motor sequencing related data of said patient and standards for functional brain activity responsive to delayed response motor sequencing tasks derived from MRI data from healthy patients;
d) detecting one or more symptoms related to Parkinson's disease in said patient based on said comparisons.

2. The process of claim 1, wherein:
said step of stimulating a patient includes the steps of visually presenting a plurality of sets of numerical cues to a patient, delaying for short periods and having said patient respond with a sequence of motor actions separately corresponding to said cues.

3. The process of claim 2, wherein:
said regions of the brain known to be affected by Parkinson's disease include the basal ganglia and SMA.

4. The process of claim 1, further including the step of:
diagnosing Parkinson's disease based on said symptoms detected in the patient.

5. The process of claim 1, further including the steps of:
analyzing said delayed response motor sequencing related MRI data by also making comparisons between said data of said patient and standards for delayed response motor sequencing functional brain activity derived from delayed response motor sequencing MRI data associated with patients known to be afflicted with Parkinson's disease; and
detecting the severity of one or more symptoms related to Parkinson's disease in said patient based on said comparisons.

6. The process of claim 5, further including the step of:
determining the staging of the Parkinson's disease based on the severity of said symptoms detected in said patient.

7. The process of claim 1, further including the steps of:
administering a medication to said patient intended to address symptoms related to Parkinson's disease;
stimulating said a patient using said delayed response motor sequencing task in order to activate said regions of the brain known to be affected by Parkinson's disease while said patient is under medication;
acquiring and recording delayed response motor sequencing related MRI data indicative of the functional MRI brain activity of the patient responsive to said delayed response motor sequencing task;
comparing the delayed response motor sequencing related MRI data acquired while said patient is off medication with said delayed response motor sequencing related MRI data acquired while said patient is on medication; and
gauging the effectiveness of said medication based on the results of comparing said data.

8. In a functional MRI scanning process in which an activation task is performed during an MRI scan for the purpose of generating functional activity data, the process including the steps comprising:
a) stimulating a patient using an delayed response motor sequencing task in order to activate regions of the brain known to be affected by Parkinson's disease;
b) acquiring and recording delayed response motor sequencing related MRI data indicative of the functional MRI brain activity of the patient responsive to said delayed response motor sequencing task;
c) analyzing said delayed response motor sequencing related MRI data by making comparisons between said data of said patient and standards for delayed response motor sequencing functional brain activity derived from delayed response motor sequencing MRI data associated with healthy patients and patients known to be afflicted with Parkinson's disease; and
d) detecting the severity of one or more symptoms related to Parkinson's disease in said patient based on said comparisons.

9. The process of claim 8, further including the step of:
determining the staging of the Parkinson's disease based on the severity of said symptoms detected in the patient.

10. The process of claim 8, further including the steps of:
administering a medication to said patient intended to address symptoms related to Parkinson's disease as the first step in said process, and
gauging the effectiveness of said medication based on the severity of the symptoms detected in said patient.

11. The process of claim 8, wherein:
said step of stimulating a patient includes the steps of visually presenting a plurality of sets of numerical cues to a patient, delaying for short periods and having said patient respond with a sequence of motor actions separately corresponding to said cues.

12. The process of claim 1 1, wherein:
said sets of cues are of varying complexity and said delay periods are of varying length.

13. The process of claim 8, further including the step of:
diagnosing Parkinson's disease based on the severity of said symptoms detected in said patient.

14. A system for detecting functional symptoms related to Parkinson's disease using an MRI scanner, comprising the steps of:
a) activating a selected region of the brain known to be affected by Parkinson's disease by having a patient perform an delayed response motor sequencing task;
b) repeatedly acquiring MRI data using an MRI scanner to produce a time image series including task-active MRI data indicative of task-activated brain activity of the patient in the selected region;
c) comparing said task active MRI data from said patient with reference data derived from a reference database including task-active MRI data from healthy subjects for delayed response motor sequencing task-activated brain activity in the selected region; and
d) detecting one or more symptoms of Parkinson's disease based on the results of comparing said patient data and reference data.

15. The system of claim 14, wherein:
said step of comparing includes selecting and adapting the reference data from said database for specific application to said patient according to the medical condition of the patient.

16. The system of claim 14, wherein:
said step of activating a selected region includes the step of visually presenting a set of numerical cues to a patient, delaying for short periods, having said patient respond with a sequence of motor actions separately corresponding to said cues.

17. The system of claim 16, wherein:
said sets of cues are of varying complexity and said delay periods are of varying length.

18. The system of claim 14, further including the step of:
diagnosing Parkinson's disease based on said symptoms detected in said patient.

19. The system of claim 14, wherein:
said step of comparing also includes comparing said task active MRI data from said patient with reference data derived from a reference database including task-active MRI data from subjects known to be afflicted with Parkinson's disease for delayed response motor sequencing task-activated brain activity in the selected region, and
said step of detecting symptoms includes detecting the relative severity of said symptoms in said patient.

20. The system of claim 19, further including the step of:
determining the staging of the Parkinson's disease based on the severity of said symptoms detected in the patient.

21. The system of claim 19, further including the steps of:
administering a medication to said patient intended to address symptoms related to Parkinson's disease, and
gauging the effectiveness of said medication based on the relative severity of the symptoms detected in said patient.

22. A system for detecting functional symptoms related to Parkinson's disease using an MRI scanner, comprising the steps of:
a) activating a selected region of the brain known to be affected by Parkinson's disease by having a patient perform an delayed response motor sequencing task;
b) repeatedly acquiring MRI data using an MRI scanner to produce a time image series including first task-active data indicative of task-activated brain activity of the patient in the selected region;
c) comparing said first task-active data from said patient with reference data derived from a reference database including task-activated data from healthy subjects and from subjects known to be afflicted with Parkinson's disease for delayed response motor sequencing task-activated brain activity in the selected region;
d) detecting the relative severity of one or more symptoms of Parkinson's disease based on the results of comparing said first patient data and reference data;
e) administering a medication to said patient for the purpose of addressing symptoms related to Parkinson's disease;
f) activating a selected region of the brain known to be affected by Parkinson's disease by having said patient perform an delayed response motor sequencing task;
g) repeatedly acquiring MRI data using an MRI scanner to produce a time image series including second task-active data indicative of task-activated brain activity of the patient in the selected region when under said medication;
h) comparing said second task-active data from said patient with reference data derived from a reference database including task-activated data from healthy subjects and from subjects known to be afflicted with Parkinson's disease for delayed response motor sequencing task-activated brain activity in the selected region;
i) detecting the relative severity of one or more symptoms of Parkinson's disease based on the results of comparing said second patient data and reference data; and
j) gauging the effectiveness of said medication based on the relative severity of the symptoms detected in the patient when under said medication and when not under said medication.

23. The system of claim 22, wherein:
said step of comparing includes selecting and adapting said reference data from said database for specific application to said patient according to the medical condition of the patient.

24. The system of claim 22, wherein:
said step of activating a selected region includes the step of visually presenting a sets of numerical cues to a patient, delaying for short periods, having said patient respond with a sequence of motor actions separately corresponding to said cues.

25. The system of claim 24, wherein:
said sets of cues are of varying complexity and said delay periods are of varying length.

26. A system for assessing functional symptoms related to Parkinson's disease using and MRI scanner, comprising the steps of:
a) activating a selected region of the brain in a patient by having the patient perform an delayed response motor sequencing task while in an MRI scanner;
b) acquiring brain activity MRI data responsive to said task for said selected region in said patient using the MRI scanner;
c) generating a patient index of task active central nervous system activity in said selected region for said patient from said MRI data;
d) comparing said index for said patient with a reference index of task active central nervous system activity derived from database data from healthy individuals for central nervous system activity responsive to said delayed response motor sequencing task;
e) detecting symptoms of Parkinson's based on the results of comparing said patient and reference indices.

27. The system of claim 26, wherein:
said step of activating a selected region includes the step of visually presenting a sets of numerical cues to a patient, delaying for short periods, having said patient respond with a sequence of motor actions separately corresponding to said cues.

28. The system of claim 26, wherein:
said sets of numerical cues are of varying complexity.

29. The system of claim 26, further including the step of:
diagnosing Parkinson's disease based on said symptoms detected in the patient.

30. A system for assessing the functional efficacy of medications for Parkinson's disease using and MRI scanner, comprising the steps of:
a) activating a selected region of the brain by having a patient perform an delayed response motor sequencing task while in an MRI scanner;
b) acquiring a first set of brain activity data responsive to said task for said selected region in said patient using the MRI scanner;
c) generating a first index of task active central nervous system activity in said selected region for said patient from said first set of data;
e) administering a medication to said patient for the purpose of addressing symptoms related to Parkinson's disease;
d) activating said selected region of the brain by having a patient perform an delayed response motor sequencing task while in an MRI scanner;
e) acquiring a second set of brain activity data responsive to said task for said selected region in said patient using the MRI scanner;
f) generating a second index of task active central nervous system activity in said selected region for said patient while under medication from said second set of data;
g) comparing said indices representing task active brain activity in said selected region while said patient is off and on said medication; and
h) determining the efficacy of said medication based on the results of comparing said indices.

31. The system of claim 30, wherein:
said step of activating a selected region includes the step of visually presenting a set of numerical cues to a patient, delaying for short periods, having said patient respond with a sequence of motor actions separately corresponding to said cues.

32. The system of claim 31, wherein:
said sets of cues are of varying complexity and said delay periods are of varying length.
